(19) United States
(12) Patent Application Publication
(10) Pub. No.: US 2025/0082899 A1
(43) Pub. Date: Mar. 13, 2025

(54) SYSTEMS AND METHODS OF ANNEALING SEGMENTS OF A REINFORCING COIL OF A CATHETER SHAFT

(71) Applicant: Merit Medical Systems, Inc., South Jordan, UT (US)

(72) Inventors: David Christian Lentz, Sandy, UT (US); Jim Mottola, West Jordan, UT (US); Nima Moazeni, South Jordan, UT (US); John Hellgeth, Murray, UT (US)

(21) Appl. No.: 18/824,597

(22) Filed: Sep. 4, 2024

Related U.S. Application Data
(60) Provisional application No. 63/581,535, filed on Sep. 8, 2023.

(51) Int. Cl. A61M 25/00

(52) U.S. Cl. CPC A61M 25/0052; A61M 2207/10

(57) ABSTRACT

The present disclosure describes systems and methods of annealing segment of a reinforcing coil of a catheter shaft. A method includes providing a liner and annealing a segment of a reinforcing wire such that the segment is between two unannealed regions of the reinforcing wire. The method also includes, after annealing the segment of the reinforcing wire, coiling the reinforcing wire over the liner to form a wire frame having a distal portion and a proximal portion. The wire is disposed more densely in the distal portion than in the proximal portion, and the segment of the reinforcing wire that is annealed is at least proximate to the distal portion of the wire frame. The method also includes securing a jacket over the wire frame and the liner.

### RELATED APPLICATIONS

This application claims priority to U.S. Patent Application No. 63/581,535, filed on Sep. 8, 2023 and titled, “SYSTEMS AND METHODS OF ANNEALING SEGMENTS OF A REINFORCING COIL OF A CATHETER SHAFT,” which is hereby incorporated by reference in its entirety.

### TECHNICAL FIELD

The present disclosure relates generally to medical sheaths including introducer sheaths and methods to manufacture introducer sheaths. More particularly, some embodiments relate to introducer sheaths with a wire frame having a distal portion with wire disposed more densely than a proximal portion of the wire frame, and with the distal portion including an annealed segment of the wire.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1A is a side view of a portion of a liner having a metal wire disposed therein, according to an embodiment.

FIG. 1B is a cross-sectional view of the portion of the liner of FIG. 1A, according to an embodiment.

FIG. 1C is a block diagram of a catheter coil winding system, according to an embodiment.

FIG. 1D is a side view of a continuous shaft including a plurality of wire frames, according to an embodiment.

FIG. 1E is a side view of a portion of a wire frame of the continuous shaft of FIG. 1D, according to an embodiment.

FIG. 1F is a cross-section view of a portion of the continuous shaft after the continuous shaft has been cut, according to an embodiment.

FIG. 2 is a perspective view of an introducer sheath with a sheath shaft including a coiled wire frame, according to one embodiment.

FIG. 3A is a perspective component view of an introducer sheath assembly, according to one embodiment.

FIG. 3B is a perspective view of the introducer sheath assembly of FIG. 3A, assembled for use.

FIG. 4 is a flow diagram of a method of manufacturing an introducer sheath, according to one embodiment.

### DETAILED DESCRIPTION

This disclosure describes medical sheaths with a reinforcement element of a wire frame having an annealed segment disposed at least proximate to a distal portion of the wire frame. The distal portion of the wire frame may have wire disposed more densely than elsewhere on the wire frame. This disclosure also describes methods to manufacture such an introducer sheath. For example, electrodes secured to two pulleys in a catheter coil winding system may be activated to apply a charge or voltage to a segment of reinforcing wire extending between the two pulleys just before the segment of reinforcing wire is coiled around a liner or elongate member. Applying the charge to the segment of reinforcing wire heats the segment of reinforcing wire effective to anneal the segment of reinforcing wire. The electrodes also may be deactivated such that the segment of reinforcing wire between the pulleys is generally the only portion of reinforcing wire that is annealed (e.g., the annealed segment is between two unannealed portions of the reinforcing wire).

Coiled metal frames may also have a desirable spring temper that both resists kinks and increases the capacity of the shaft to temporarily elastically deform, then spring back without creating a permanent kink. This, in turn, reduces instances where a kinked or deformed introducer shaft must be removed and replaced during a therapy. The annealed segment of the reinforcing wire may form at least some of a distal portion of a coiled wire frame. This annealed segment reduces a tendency of the coiled wire frame to unravel along the annealed portion, while the coiled wire frame maintains a desirable spring temper along the unannealed portions. Also described herein are systems and methods for annealing only selected segments of a reinforcing wire immediately before the reinforcing wire is coiled around a liner, while portions of the reinforcing wire adjacent to the annealed segment remain unannealed.

In some embodiments, the wire frame may include a coiled wire frame with a distal portion having the wire coiled back over the distal portion of the coiled wire frame such that there are two portions of coiled wire overlapping one another at the distal portion. In another example, the wire frame may include a braided wire frame where the wire is braided more densely (e.g., more picks/inch) at the distal portion than elsewhere on the wire frame. According to many embodiments, the wire frame includes a radiopaque material, and the distal portion having the wire more densely disposed therein (e.g., double coiled or greater picks/inch braid) may be function as a radiopaque marker during use of the introducer sheath. For example, the distal portion of the wire frame may be used for fluoroscopy identification. While embodiments herein refer to introducer sheaths, the same features may be included on other types of sheaths such as guiding sheaths.

Furthermore, as illustrated and discussed herein, wire frames within the scope of this disclosure may be understood as segments or portions of a longer coil, wire, braid, or mesh that are continuously and serially positioned and configured to be severed from adjacent wire frames. In other words, wire frames may include repeating patterns or segments of coils or other reinforcing members that cut into segments for inclusion in discrete medical or separate medical devices.

Introducer sheaths are used in a variety of diagnostic and therapeutic procedures to provide access to a patient's vascular system. When an introducer sheath is placed in the vasculature, the introducer sheath may facilitate exchange of guidewires, catheters, contrast media, and various fluids while providing access to the vasculature and a hemostatic seal.

To facilitate the exchange of medical tools and fluids, the introducer sheath includes a hub configured to remain exterior to the patient's skin. The hub forms a chamber that may be accessed through various ports. For example, a hub may include a side port and an introducer bore. In some embodiments, the side port is fluidly coupled to a fluid channel that is controlled by a stop-cock. The fluid channel conveys fluids or medicaments to and from the hub. A practitioner may introduce guidewires, catheters, stents, balloons, and other articles and/or materials to be introduced into the patient through the introducer bore. A valve or a seal may maintain hemostasis of the introducer sheath while allowing a medical instrument to be introduced through the introducer bore into the chamber. The hub chamber is in fluid communication with a sheath shaft. The sheath shaft is inserted into the vasculature of the patient, and provides through the skin to the vasculature.

A problem with current sheaths is vascular access bleeding that sometimes occurs after the sheath shaft has been removed. In general, there is a relationship between the size of the outer diameter of the inserted sheath shaft and the risk of bleeding complications. Thus, sheaths shafts with thinner walls correlate to a decrease in the size of the outside diameter (and therefore a decrease in the size of the hole at the vascular entry site) without decreasing the size of the inside diameter of the sheath. Thus, thin walled sheaths may reduce bleeding complications when compared to thicker wall sheaths with the same inside diameter.

However, simply reducing the thickness of the walls of a sheath shaft introduces additional concerns. Specifically, with thinner walls, there may be a greater concern of kinking and deformation from a cylindrical shape. When a sheath shaft kinks, the passageway to the vascular system of a patient may be blocked. If a sheath shaft does not maintain its shape, medical instruments may not fit.

To maintain the shape of a thin walled sheath shaft, the wall of the shaft may be reinforced. A polymer shaft may be reinforced with a metal reinforcing element, such as the coiled metal frame. The reinforcing element may thus increase the strength, stiffness, burst strength, creep resistance, and other properties of the shaft.

In some embodiments, an introducer shaft is coupled to a hub at a proximal end. The shaft forms a lumen that is in fluid communication with the chamber of the hub. In embodiments having a coiled wire frame, at least the proximal portion of the coiled wire frame may be unannealed (e.g., free from annealing). An outer polymer member such as a jacket may encompass the coiled wire reinforcing element, and an inner polymer member such as a liner may form an inside surface of the wall of the shaft.

Introducer sheaths having wire more densely disposed in the distal portion of a wire frame may be manufactured using the methods described in more detail below. In some embodiments, one or more metal wires are coiled or wound on a spring winder from a proximal end to a distal end, then further coiled or wound from the distal end only partially towards the proximal end to form the distal portion having wire more densely disposed than elsewhere in the wire frame. In many embodiments, a liner and a jacket are reflowed or melt-bonded onto the frame, and a hub is overmolded around a proximal portion of the reflowed shaft. These methods may be done along a length sufficient to produce a plurality of introducer sheaths. For example, in some embodiments, a larger amount of wire may be coiled along a nylon core to form a frame. The nylon core may be heated and stretched to reduce the diameter of the nylon core for removal of the frame. A hub may be overmolded or otherwise secured around a proximal portion of the sheath shafts.

The phrase “coupled to” is broad enough to refer to any suitable coupling or other form of interaction between two or more entities, including mechanical, fluidic and thermal interaction. Thus, two components may be coupled to each other even though they are not in direct contact with each other. The phrase “fluid communication” is used in its ordinary sense, and is broad enough to refer to arrangements in which a fluid (e.g., a gas or a liquid) can flow from one element to another element when the elements are in fluid communication with each other.

The terms “proximal” and “distal” are opposite directional terms. As used herein, the distal end of a device or component is the end of the component that is furthest from the physician during ordinary use. The proximal end refers to the opposite end, or the end nearest the physician during ordinary use. For example, the proximal end of an introducer sheath used in minimally invasive vascular treatment is the end accessible to a practitioner during use, while the distal end is disposed within a patient's vascular system when the sheath is placed into such a patient.

An assembler may be any person, system, or machine used in the manufacture of the introducer sheaths.

Embodiments may be understood by reference to the drawings, wherein like parts are designated by like numerals throughout. The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

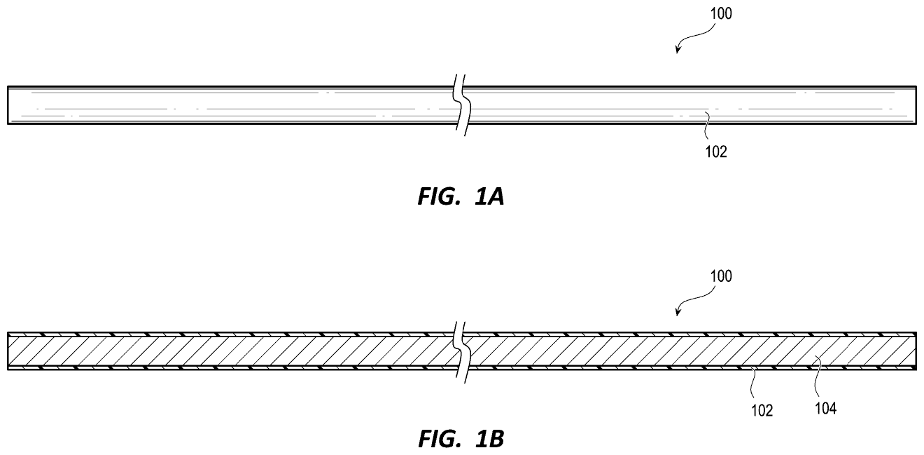

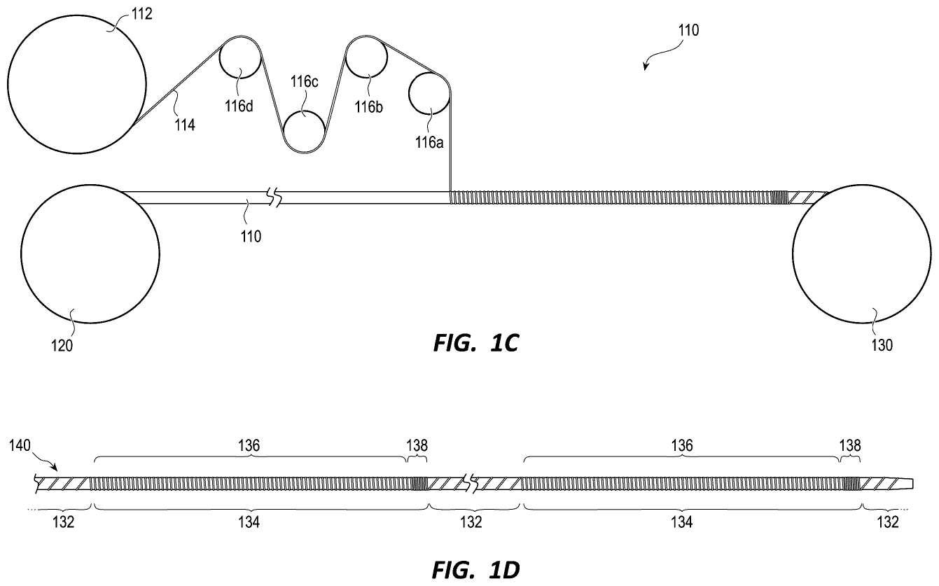

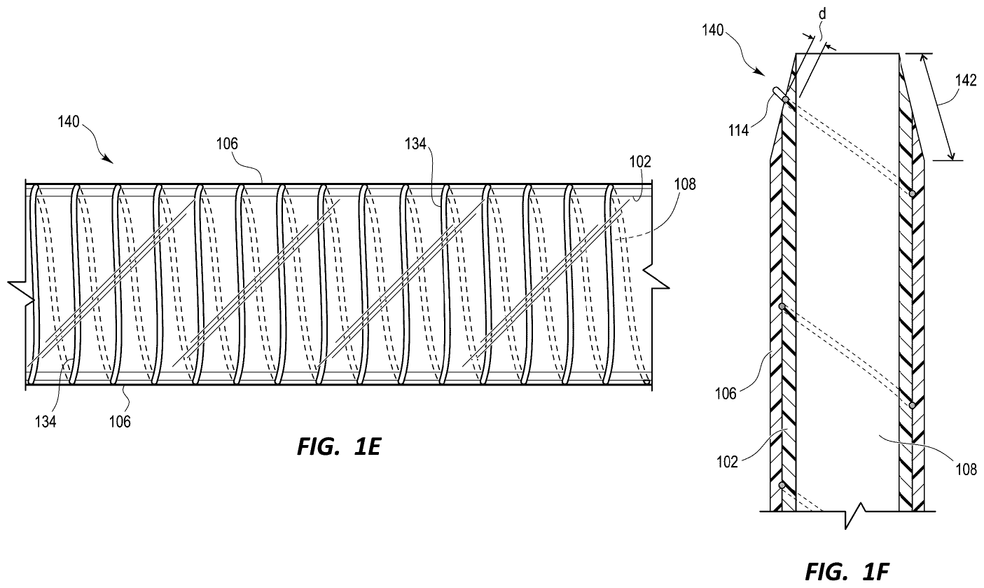

Turning now to the drawings, FIG. 1A is a side view of a portion of an elongate member 100 including a liner 102 having a metal wire 104 disposed therein, according to an embodiment and FIG. 1B is a cross-sectional view of the portion of the liner of FIG. 1A. FIG. 1C is a simplified block diagram of a catheter coil winding system 110, according to an embodiment. FIG. 1D is a side view of an elongate member 100 including a plurality of wire frames 134 formed from the catheter coil winding system 110. FIG. 1E is a side view of a portion of a wire frame 134 of a continuous shaft 140 of FIG. 1D, according to an embodiment. FIG. 1F is a cross-section view of a portion of the continuous shaft 140 after the continuous shaft 140 has been cut, according to an embodiment.

Referring specifically to FIG. 1C, in many embodiments, a catheter coil winding system 110 is configured to anneal selected segments of reinforcing wire 114 pulled from a spool 112 just prior to (e.g. immediately before) the reinforcing wire 114 is wound around the elongate member 100 including the liner 102. In other words, the catheter coil winding system 110 may include an “in-line” annealing system. The catheter coil winding system 110 may include a spool holder configured to hold the spool 112 of the reinforcing wire 114.

The reinforcing wire 114 may include tempered steel or brass wires configured to provide a desired spring temper induced to increase their upper limit of elasticity in the coiled wire frame 134. In some embodiments, steels used in the reinforcing wire 114 may include stainless steel, low-alloy, medium-carbon steel, nitinol, and high-carbon steel including those with high yield strength. The spring temper of the reinforcing wire 114 may allow the coiled wire frame 134 to return to its original shape despite deflection, deformation, and/or twisting. In some embodiments, the reinforcing wire 114 may include flat wires. In other embodiments, the reinforcing wire may include round wires.

The catheter coil winding system 110 also may include an annealing assembly configured to selectively anneal segments of the reinforcing wire between the spool holder and the elongate member 100. The annealing assembly described herein is not limited to the catheter coil winding system 110 shown in FIG. 1C, but may be included in any catheter coil winding system without departure from the scope of this disclosure. In some embodiments, the annealing assembly includes one or more pulleys 116a-d positioned between the spool holder (e.g., the spool 112 of reinforcing wire 114) and the elongate member 100. The one or more pulleys 116a-d are positioned and configured to have the reinforcing wire 114 wind around a portion of each of the one or more pulleys 116a-d before being coiled around the elongate member 100. In the catheter coil winding system 110 shown in FIG. 1C, the catheter coil winding system 110 includes four pulleys 116a-d positioned and configured to have the reinforcing wire 114 wind around a portion of each of the four pulleys 116a-d. Other embodiments, however, may include fewer (e.g., one, two, or three) pulleys or more (e.g., five, six, seven, and so on) pulleys than the four pulleys 116a-d shown in FIG. 1C.

In some embodiments, the one or more pulleys 116a-d may be at least partially metal. For example, the one or more pulleys 116a-d may include a rotatable metal disc or cylinder that the reinforcing wire 114 at least partially contacts. The catheter coil winding system 110 also may include an electrode secured to at least one pulley of the one or more pulleys 116a-d and an actuator configured to activate and deactivate the electrode. In some embodiments, the catheter coil winding system 110 includes two electrodes each secured to a different pulley of the pulleys 116a-d. For example, the catheter coil winding system 110 may include a first electrode secured to a first pulley 116a and a second electrode secured to a second pulley 116b. In some embodiments, the catheter coil winding system 110 may include multiple electrodes each coupled a different pulley of the multiple pulleys 116a-116d.

As noted above, the actuator is configured to selectively activate and deactivate the electrodes. In some embodiments, the electrode may be secured to a pulley of the multiple pulleys 116a-d such that the electrode applies a voltage or charge to the metal disc or cylinder of the pulley when the electrode is activated. Activating the electrodes is effective to anneal a segment of the reinforcing wire 114 before the segment of the reinforcing wire is coiled around at least the portion of the elongate member 100 (e.g., after the reinforcing wire 114 has been unspooled from the spool 112, but before the reinforcing wire 114 has been wound around the elongate member 100). For example, when the catheter coil winding system 110 include the first electrode secured to the first pulley 116a and the second electrode secured to the second pulley 116b, activating the electrodes secured to the first pulley 116a and the second pulley 116b anneals the segment of the reinforcing wire 114 that extends between the first pulley 116a and the second pulley 116b. More specifically, activating the electrodes secured to the first pulley 116a and the second pulley 116b may apply a voltage or charge to the segment of the reinforcing wire 114 positioned between the first pulley 116a and the second pulley 116b effective to heat and anneal the segment of the reinforcing wire 114 that extends between the first pulley 116a and the second pulley 116b.

In some embodiments, the two pulleys (e.g., pulleys 116a, 116b) having an electrode secured thereto are positioned at a selected and/or adjustable linear distance such that the activation of the electrodes secured to the two pulleys anneals a segment of the reinforcing wire 114 have a selected distance corresponding or correlated to the linear distance between the two pulleys. In embodiments having more than two pulleys, the two pulleys having an electrode secured thereto may be the last two pulleys the reinforcing wire 114 contacts before the reinforcing wire 114 is would around the elongate member 100.

Turning to FIG. 1D, the reinforcing wire 114 may be coiled around the elongate member 100 effective to form one or more coiled wire frames 134 on the elongate member 100. In the embodiment shown in FIG. 1D, multiple coiled wire frame 134 are coiled around the elongate member 100. In other embodiments, only a single coiled wire frame may 134 be coiled around the elongate member 100. The coiled wire frame 134 may include a proximal portion 138 and a distal portion 136 having the reinforcing wire 114 coiled more densely and/or more tightly the proximal portion 138.

The catheter coil winding system 110 may be configured such that the at least some (e.g. most or all) of the distal portion 136 includes the annealed reinforcing wire 114. For example, in some embodiments, the annealed segment is located at the distal end of the distal portion 136 when coiled on the elongate member 100. Annealing at least some of the distal portion 136 removes residual stresses and the spring temper of the coiled wire frame 134 along the annealed portion of the distal portion 136. For example, annealing may reduce the tendency of the coils to straighten out, tending to prevent unraveling. In some embodiments, the catheter coil winding system 110 is configured to automatically activate and deactivate the electrodes on the first pulley 116a and the second pulley 116 during coiling of the reinforcing wire 114 and formation of the coiled wire frame 134 such that the annealed segment forms at least some of the distal portion 136 of the coiled wire frame 134.

In some embodiments the annealed segment at the distal end of the coiled wire frame 134 may be approximately 1/32 inches long to approximately ¼ inches long, including about 1/16 inches long to about 3/16 inches long and about ⅛ inches long. The length of the annealed portion may vary based on application. In some embodiments, the annealed portion may be approximately 1/32 inches long to approximately ¼ inches long, including about 1/16 inches long to about 3/16 inches long and about ⅛ inches long. In some embodiments, a plurality of portions on the length of a long coiled wire frame 134 may be annealed. The long coiled wire frame 134 may subsequently be cut to form multiple coiled wire frames for inclusion in introducer sheath shafts. The positions of the plurality of annealed portions along the coiled metal frame may thus ultimately correspond to portions that will be disposed as distal ends of a plurality of introducer sheath shafts.

With the distal portion 136 being coiled more tightly than the proximal portion 138, the distal portion 136 includes wire more densely disposed (e.g., tighter pitch) than the proximal portion 138 (e.g., wider pitch). The coiled wire frame 134 includes may include radiopaque material. For example reinforcing wire 114 may include one or more of tungsten, palladium, platinum, gold, tantalum, depleted uranium, or high radiopaque wire that may be coiled into the coiled wire frame 134 for fluoroscopy identification. In many embodiments, the coiled wire frame 134 consists or consists essentially of the radiopaque material. Accordingly, the distal portion 136 may include more densely disposed radiopaque strands of material than the proximal portion 138 of the coiled wire frame 134. The more densely disposed radiopaque strands of material in the distal portion 136 act as a radiopaque marker in fluoroscopy identification, thereby allowing a user to more easily identify the distal region of a sheath shaft 232, 312 during use.

In some embodiments, the distal portion 136 includes a longitudinal length of less than about 125 mm, less than about 100 mm, less than about 75 mm, less than about 50 mm, less than about 25 mm, less than about 10 mm, less than about 5 mm, less than about 3 mm, less, than about 2 mm, less than about 1 mm, about 1 mm to about 125 mm, about 1 mm to about 5 mm, about 5 mm to about 10 mm, about 10 mm to about 25 mm, about 25 mm to about 50 mm, about 50 mm to about 100 mm, about 100 to about 150 mm, about 1 mm, about 2 mm, about 3 mm, about 4, about 5, about 10 mm, about 25 mm, about 50 mm, about 75 mm, or about 100 mm.

In some embodiments, a coiled wire frame (not shown) may include multiple portions having a more dense coil and/or multiple portions having a less dense coil. For example, the wire frame 134 may include the distal portion 136, a first intermediate portion, a second intermediate portion, and the proximal portion 138. The first intermediate portion may be disposed between (e.g. adjacent to) the distal portion and the second intermediate portion, and may include a less dense coil than the coil of the distal portion 136 and the second intermediate portion. The second intermediate portion may be disposed between (e.g., adjacent to) the first intermediate portion and the proximal portion 138, and may include a more dense coil than the first intermediate portion and the proximal portion 138.

In these and other embodiments, the distal portion 136 may extend a longitudinal length of less than about 2 mm, the first intermediate portion adjacent the distal portion 136 may extend a longitudinal length of about 3 mm to about 7 mm, the second intermediate portion between first intermediate portion and the proximal portion 138 may extend a longitudinal length about less than about 2 mm.

In use, the distal portion 136 and the second intermediate portion having the more dense coil would be visible under fluoroscopy as distinct from the first intermediate portion and the proximal portion 138 having the less dense weave(s). The distance between the distal portion 136 and the second intermediate portion (e.g., the longitudinal length of the first intermediate portion) may be known by the operator and used to measure anatomical features such as a lesion or vessel aneurysm length. Any number or combination of portions with more dense coils may be used. For example, a single device may have two, three, four, or more dense portions that may or may not be distributed at even distances from each other. These portions can be configured for measurement of anatomical features, identification of portions of the introducer sheath 100, or both.

The annealing assembly disclosed herein may be applied to any systems and/or methods for manufacturing an introducer sheath comprising a coiled wire frame. For example, the annealing assembly disclosed herein may be utilized in systems and methods having an assembler that extrudes a nylon core and coils the reinforcing wire 114 over the core effective form a coiled wire frame. In some embodiments, the coiled wire frame may be coiled by winding the metal (e.g., wire) on a spring winder. The annealing assembly may be applied to systems and methods wherein the reinforcing wire 114 is doubled over a distal portion of the coiled wire frame when coiling the reinforcing wire such that having the metal disposed more densely in the distal portion than in the rest (e.g., proximal portion) of the metal frame. At least some (e.g., most or all) of the distal portion may include the annealed reinforcing wire 114. In some embodiments that include the annealing assembly disclosed herein, the assembler may then heat and stretch the core and remove the coiled wire frame 134 including annealed segment. In some embodiments, the coiled wire frame 134 may be formed without formation and removal of the nylon core, and may be instead before formed on a spring winder. Systems and methods including the annealing assembly disclosed herein may be configured to extrude a liner and the slide the liner over a PTFE-coated mandrel before the coiled wire frame 134 is slide over the liner. The coiled wire frame 134 may then be twisted down, and any excess coil at the proximal end of the coiled wire frame 134 may be trimmed. A jacket and an FEP heat shrink shell may then be extruded and slid (individually or simultaneously) over top of the coiled wire frame 134. This assembly may then be exposed to heat, causing the liner and the jacket to reflow. The FEP shell may then be removed, and the resulting assembly may be removed from the mandrel.

In some embodiments, the annealing assembly disclosed herein may be utilized with a catheter coil winding system 110 configured to form multiple wire frames 134 on a single elongate member 100. Turning back in the drawings to FIGS. 1A and 1B, the elongate member 100 includes a liner 102 having a metal wire 104 disposed therein, according to an embodiment. The material of the liner 102 may comprise polyamide resins configured to seal the interior surface of the coiled wire frame 134. The liner 102 can include a surface comprising a lubricious polymeric material. For example, the material can comprise any bio-compatible material having low frictional properties (e.g., TEFLON®, PTFE, fluorinated ethylene propylene (FEP), polyethylene, polyamide, ethylene chlorotrifluoro-ethylene, ethylene tetrafluoroethylene, PVDF).

The metal wire 104 may include a stretchable wire that acts as a mandrel. For example, the metal wire 104 may include a copper wire, such as a silver-plated copper wire. In some embodiments, the metal wire 104 may be coated with low friction materials to ease loading and removal of the liner 102 and coiled wire frame 134. For example, in some embodiments, the low surface friction mandrel may be a polytetrafluoroethylene (PTFE)-coated metal wire 104 to slide a stainless steel metal frame onto. In some embodiments, lubricants may be used to reduce the friction on the surface of the low surface friction metal wire 104.

Turning now to FIGS. 1C and 1D, in some embodiments, the catheter coil winding system 110 is configured to coil a plurality of wire frames 134 on a single, continuous elongate member 100. As previously noted, FIG. 1C is a block diagram of the catheter coil winding system 110 that schematically shows a process for coiling the reinforcing wire 114 around the elongate member 100, according to an embodiment.

The catheter coil winding system 110 may include a spool holder configured to hold a first spool 120 of the elongate member 100 that includes the liner 102 having the metal wire 104 disposed therein. The first spool 120 may include a single, continuous elongate member 100 rolled onto or otherwise wrapped around the spool 120. In some embodiments, the first spool 120 is positioned or positionable on the spool holder of the catheter coil winding system 110 such that the elongate member 100 may be unrolled from the first spool 120 to extend past the one or more pulleys for coiling of the reinforcing wire 114 thereon.

In some embodiments, the catheter coil winding system 110 includes an assembly 111 including the spool 112 of reinforcing wire 114 and the one or more pulleys 116a-d. The assembly 111 is configured to rotate around the elongate member 100 coil the reinforcing wire around the elongate member 100. In other words, the catheter coil winding system 110 is configured such that one or more (e.g., all) of the spool 112 of reinforcing wire 114 and the one or more pulleys 116a-d rotate around the elongate member 100 to coil the reinforcing wire 114 around at least a portion of the elongate member 100.

In some embodiments, as one or more (e.g., all) of the spool 112 of reinforcing wire 114 and the one or more pulleys 116a-d rotate around the elongate member 100 to coil the reinforcing wire on the elongate member 100, the elongate member 100 may be pulled from the first spool 120 at a first rate when the proximal portion 138 is being formed and a second rate slower than the first rate when the distal portion 136 is being formed. Pulling the elongate member 100 from the first spool 120 at the first rate and the second rate is effective for form the distal portion 136 having the reinforcing wire 114 coiled more densely thereon than the proximal portion 138 of the wire frame 134.

In some embodiments, as one or more (e.g., all) of the spool 112 of reinforcing wire 114 and the one or more pulleys 116a-d rotate around the elongate member 100 to coil the reinforcing wire on the elongate member 100, the elongate member 100 may be pulled from the first spool 120 at a third rate to form an interim portion 132 of reinforcing wire 114 on the elongate member 100. The third rate may be faster than the first rate and the second rate such that the reinforcing wire 114 is coiled less densely (e.g., wider pitch) than both the proximal portion 138 and the distal portion of the coiled wire frame 134. The interim portion 132 may be coiled between the distal portion 136 of a first wire frame 134′ of the plurality of wire frames and a proximal portion 138 of a second wire frame 134″ of the plurality of wire frames adjacent to the first wire frame 134′. The positioning of the interim portion 132 between the first wire frame 134′ and the second wire frame 134″ connects the first wire frame 134′ to the second wire frame 134″ and also spaces the distal portion 136 of the first wire frame 134′ from the proximal portion 138 of the second wire frame 134″.

In some embodiments, the elongate member 100 is secured to a second spool 130 distal to the first spool 120. The first spool 120 and the second spool 130 may be positioned such that reinforcing wire 114 pulled from the spool 112 may be coiled around the elongate member 100 extending between the first spool 120 and the second spool 130. For example, the pulley 116a may be positioned between the first spool 120 and the second spool 130. The second spool 130 may be positioned on a second spool holder configured to rotate the second spool 130 around an axis of the second spool 130 effective to pull the elongate member 100 from the first spool 120. More specifically, the second spool holder may be configured to rotate the second spool 130 around the axis of the second spool 130 at different rates effective pull the elongate member 100 from the first spool 120 at the first rate, the second rate, and/or the third rate to form the proximal portion 138, the distal portion 136, and the interim portion 132 on the elongate member 100. The elongate member 100 may be secured to second spool 130 such that as the second spool holder rotates the second spool 130, the elongate member 100 having the plurality of coiled wire frames 134 is rolled around the second spool 130.

In some embodiments, the catheter coil winding system 110 may include a proximal chuck rather than the first spool 120 and/or a distal chuck rather than the second spool 130. In other words, in some embodiments, the circle 120 represents a proximal chuck and/or the circle 130 represents a distal chuck. The proximal chuck and the distal chuck may be configured to secure to the elongate member 100 and may be spaced from one another at a distance for a plurality of wire frames to be formed on the elongate member 100 between the proximal chuck and the distal chuck. The proximal chuck and/or the distal chuck may be configured to rotate the elongate member 100 to coil the reinforcing wire 114 around the elongate member 100. In some embodiments, the proximal chuck and/or the distal chuck are configured to move simultaneously as the reinforcing wire 114 is being coiled around the elongate member 100 effective to form the proximal portion 138, the distal portion 136, and the interim portion 132. For example, with the elongate member 100 being secured thereto, the proximal chuck and/or the distal chuck may be configured to move the elongate member 100: at a first rate as the reinforcing wire 114 is being coiled around the elongate member 100 to form the proximal portion 138; at a second rate slower than the first rate as the reinforcing wire 114 is being coiled around the elongate member 100 to form the distal portion 136; and at a third rate faster than the first rate and the second rate as the reinforcing wire 114 is being coiled around the elongate member 100 to form the interim portion 132. In some embodiments of the catheter coil winding system 110, the proximal chuck may replace the first spool holder/first spool 120, while the second spool holder/second spool 130 remains. In some embodiments, the distal chuck may replace the second spool holder/second spool 130, while the first spool holder/first spool 120 remain.

Turning to FIGS. 1E and 1F, the catheter coil winding system 110 may be configured to dispose a jacket 106 over the coiled wire frame 134 and the liner 102. In some embodiments, the metal wire 104 may be removed from the liner 102 prior to the jacket 106 being disposed over the liner 102. In some embodiments, the jacket 106 may be disposed over the liner 102 while the metal 104 extends through the liner 102. The jacket 106 may have a hydrophilic coating and a larger diameter than the coiled wire frame 134.

The catheter coil winding system 110 also may be configured to dispose an FEP shell (not shown) over the jacket 106. In some embodiments, the metal wire 104 may be removed from the liner 102 prior to the FEP shell being disposed over the jacket 106. In some embodiments, the FEP shell may be disposed over the jacket 106 while the metal 104 extends through the liner 102. The FEP shell may have a larger diameter than the jacket 106 and encompass the jacket 106, the coiled wire frame 134, and the liner 102.

The catheter coil winding system 110 may be configured to apply heat to melt or reflow the liner 102 and the jacket 106. Heating the FEP shell causes it to shrink, reducing its diameter. The reduced diameter of the FEP shell causes the sidewalls of the FEP shell to apply pressure to the encompassed elements (i.e., the jacket 106, the coiled wire frame 134, and the liner 102). The reflow temperature is within a range that causes the jacket 106 and liner 102 to melt, but not the FEP shell. Thus, the jacket 106 and liner 102 are reflowed to the coiled wire frame 134 to create a composite conduit forming a sheath shaft 140.

According to various embodiments of the catheter coil winding system 110, the jacket 106 may be applied to the liner 102 and the plurality of coiled wire frames 134 before or after the liner 102 and the plurality of coiled wire frames 134 are rolled around the second spool 130. According to various embodiments of the catheter coil winding system 110, the FEP shell may be applied to the jacket 106 before or after the liner 102 and the plurality of coiled wire frames 134 are rolled around the second spool 130.

When the metal wire 102 is removed from the liner 102 (whether before or after the jacket 106 is applied to the liner 102 and the plurality of wire frames 134), a sheath shaft lumen 108 is formed in the shaft 140. The shaft 140, then may include the liner 102 defining the sheath shaft lumen 108, the jacket 106, and the coiled wire frame 134 disposed between the jacket 106 and the liner 102. In some embodiments, the shaft 140 may include a continuous shaft that includes a plurality of wire frames 134 disposed between the jacket 106 and the liner 102 with the interim portion 132 separating adjacent wire frames of the plurality of wire frames 134. While the liner 102, the coiled metal frame 134, and the jacket 106 are shown as distinct layers in FIGS. 1E and 1F, the polymer materials of the liner 102 and the jacket 106 may be melted and reflowed together, bonding to the coiled wire frame 134 and each other and filling any openings in the coiled wire frame 134.

Accordingly, the catheter coil winding system 110 may be configured to form a continuous sheath 140 (e.g., continuous shaft) comprising a liner 102, a plurality of coiled wire frames 134, an interim portion 132 between adjacent coiled wire frames of the plurality of coiled wire frames 134, and a jacket 106 secured over the plurality of coiled wire frames 134 and the interim portion 132 of the reinforcing wire 114. The plurality of wire frames 134 of the continuous sheath 140 may be coiled around the liner 102. Each wire frame of the plurality of wire frames 134 may include the proximal portion 138 and the distal portion 136 having the reinforcing wire 114 coiled more densely than the proximal portion 138. The interim portion 132 of the reinforcing wire 114 may be coiled at least partially around the liner 102 and connect the distal portion 136 of a first wire frame 134′ of the plurality of wire frames 134 to the proximal portion 138 of a second wire frame 134″ of the plurality of wire frames 134 adjacent to the first wire frame 134′. The distal portion 136 of the first wire frame 134′ may be spaced from the proximal portion 138 of the second wire frame 134″. The continuous sheath 140 may include a plurality of annealed segments of the reinforcing wire 114, each annealed segment of the reinforcing wire 114 being disposed at least proximate to the distal portion 136 of a coiled wire frame of the plurality of wire frames 134.

In some embodiments, the catheter coil winding system 110 may be configured such that the continuous shaft 140 is rolled on the second spool 130 (e.g., after application of the jacket 106 and/or after the metal wire 104 is removed from the liner 102). The continuous shaft 140 may then be selectively unrolled from second spool 130. In these and other embodiments, a user may cut the continuous shaft 140 at an interim portion 132 between two adjacent coiled wire frames 134 in the continuous shaft to produce a single first sheath 140′ including only one wire frame 134 having the proximal portion 138 and the distal portion 136. In some embodiments, the metal wire 104 may be removed from the liner 102 after the single first sheath 140′ is cut from continuous shaft 140. For example, after cutting the first sheath 140′ from the continuous shaft 140, the metal liner 104 may be stretched and pulled from the first sheath 140′ to remove the metal liner 104 and form the lumen 108 in the first sheath 140′.

Turning specifically to FIG. 1F, after cutting the single sheath 140′, one or both end regions 142 of the single sheath 140′ may be tapered (e.g. necked down) for securing to other components. For example, at least the jacket 106 may be tapered at one or both end regions 142 of the single sheath 140′. In some embodiments, tapering the single sheath 140′ may result in a portion of reinforcing wire 114 from the coiled wire frame 134 or the interim portion 132 protruding from the single sheath 140′. For example, a portion of reinforcing wire 114 may protrude from between the liner 102 and the jacket 106 at one or more both end regions 142.

Accordingly, in many embodiments, a portion of the reinforcing wire 114 may be removed from one or both end regions 142 of the single sheath 140′ after the single sheath 140′ is cut from the continuous shaft 140. For example, a laser may be applied to the reinforcing wire 114 at the end region 142 to laser cut or ablate the reinforcing wire 114 in the wire frame 134 or the interim portion 132. Applying a laser to the reinforcing wire 114 cut remove the reinforcing wire a distance d of about 0.5 mm to about 2 mm into the single sheath 140′.

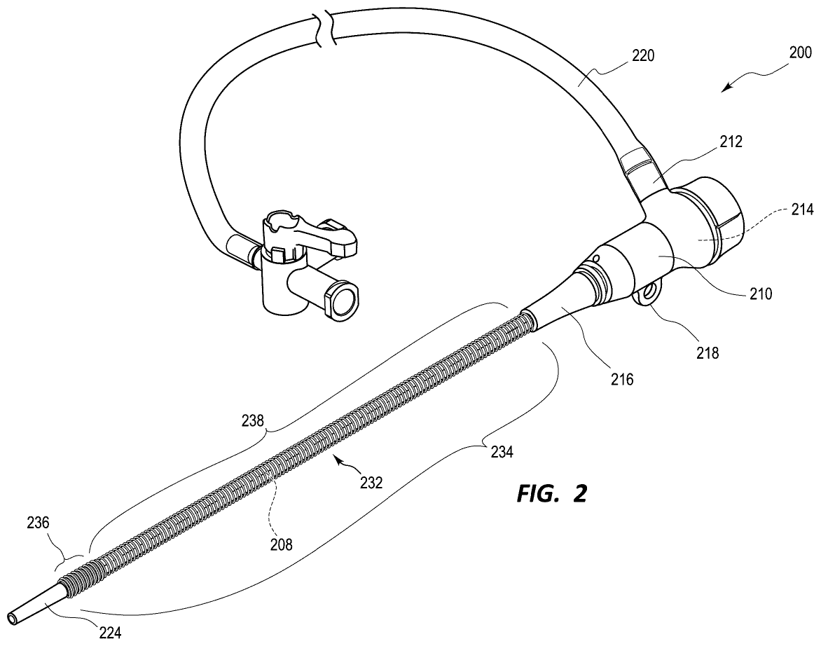

Turning in the drawings to FIG. 2, an introducer sheath 200 having a sheath shaft 232 formed according to any embodiments of the catheter coil winding system 110 described above. For example, the sheath shaft 232 may include a coiled wire (e.g. metal) frame 234 having a distal portion 236 and a proximal portion 238, with wire of the wire frame 234 being disposed more densely in the distal portion 236 than the proximal portion 238. The wire frame 234 may include an annealed segment, and may be disposed between a liner 102 and a jacket 106. The sheath shaft 232 may be formed according to any of the systems and/or methods described herein.

The sheath shaft 232 may be coupled to and in fluid communication with the hub 210. During some procedures, the hub 210 may be is intended to remain exterior of a patient, and the sheath shaft 232 is intended to at least partially be placed within the vascular system of the patient. The hub 210 forms a chamber that may be accessed via a side port 212 or an introducer bore 214. A suture ring 218 is coupled to the hub 210 and provides a mechanism allowing a practitioner to grasp the introducer sheath 200 while allowing the introducer sheath 200 to be sutured or fastened to the patient once the introducer sheath 200 has been properly placed. The side port 212 and the introducer bore 214 provide entry for medical devices and fluids. For example, a physician may insert a dilator into the introducer bore 214 to assist with placing the introducer sheath 200. The dilator enters the introducer bore 214 through a seal or a valve that maintains hemostasis when the introducer sheath 200 in in communication with the vasculature. Similarly, a fluid channel 220 may couple to the side port 212, establishing a fluid passageway with the chamber of the hub 210. In some embodiments, a sleeve may be used to swage the fluid channel 220 onto the side port 212. A sleeve may be placed over the fluid channel 220 onto the side port 212. Any medical instrument or fluids that enter the chamber of the hub 210 may continue through an opening at the hub distal end 216 into a sheath shaft lumen 108. Thus, the side introducer bore 214 and a lumen of the fluid channel 220 may both be in fluid communication with the sheath shaft lumen 108.

A tip 224 may be coupled to the sheath shaft 232. A cuff (not shown) may overlap the tip-shaft joint to strengthen the joint. The sheath shaft 232 may include the coiled wire frame 234 with an exterior and interior surface coated with a polymer material. In the illustrated embodiment, the liner 102 is coupled to the interior surface of the coiled wire frame 134 and defines the inside surface of the sheath shaft 232 and a jacket 106 is coupled to the exterior surface of the coiled wire frame 234 and defines the outside surface of the sheath shaft 232.

The introducer sheath 200 may have a thinner wall when compared with traditional introducer sheaths. In some embodiments, the introducer sheath 200 may have an

$$\frac{inner diameter lower limit}{outer diameter upper limit}$$

ratio of greater than 0.85. For example, an introducer sheath for a 4 French (4 F) needle or catheter may have an

$$\frac{inner diameter lower limit}{outer diameter upper limit}$$

ratio of greater than 0.85, an introducer sheath for a 5 F needle or catheter may have an

$$\frac{inner diameter lower limit}{outer diameter upper limit}$$

ratio of greater than 0.87, an introducer sheath for a 6 F needle or catheter may have an

$$\frac{inner diameter lower limit}{outer diameter upper limit}$$

ratio of greater than 0.89, an introducer sheath for a 7 F needle or catheter may have an

$$\frac{inner diameter lower limit}{outer diameter upper limit}$$

ratio of greater than 0.90. In some embodiments, the introducer sheath 200 may have an

$$\frac{inner diameter}{outer diameter}$$

ratio ranging between 0.87 and 0.93. For example, an introducer sheath for a 4 F needle or catheter may have an average

$$\frac{inner diameter}{outer diameter}$$

ratio ranging between 0.87 and 0.89, an introducer sheath for a 5 F needle or catheter may have an average

$$\frac{inner diameter}{outer diameter}$$

ratio ranging between 0.89 and 0.90, an introducer sheath for a 6 F needle or catheter may have an average

$$\frac{inner diameter}{outer diameter}$$

ratio ranging between 0.90 and 0.91, and an introducer sheath for a 7 F needle or catheter may have an average ratio ranging

$$\frac{inner diameter}{outer diameter}$$

between 0.92 and 0.93.

At least a portion of the wire frame 234 may be annealed and at least a portion of the wire frame 234 may be unannealed. In some embodiments, at least some of the distal portion 236 is annealed and the proximal portion 108 is unannealed. Various aspects and positioning of annealing a wire frame of a sheath are disclosed in U.S. patent application Ser. No. 17/457,878, the disclosure of which is incorporated herein, in its entirety, by this reference. The term unannealed refers to portions of the coiled metal frame 234 that retain or otherwise are configured with more spring temper than an annealed portion. The tip 224 may be coupled to the sheath shaft 232 adjacent an annealed portion of the distal portion 236. In some embodiments, a cuff may be positioned across the joint between the tip 224 and the annealed portion of the distal portion 236. The cuff may smooth the transition between the tip 224 and the annealed portion of the distal portion 236 and increase the strength of the joint.

The sheath shaft 232 may include the tip 224 coupled to the composite conduit sheath shaft 232 adjacent the distal tip of the coiled wire frame 234. The tip 224 may comprise a polymeric material configured to be malleable to reduce trauma when the introducer sheath enters the vascular system and increase trackability of the introducer over a guidewire. A butt joint may couple the tip 224 to the sheath shaft 232. A butt joint ensures smoothness through the transition from the tip 224 to the sheath shaft 232. In some embodiments, the tip 224 may overlap the sheath shaft 232. In some embodiments, a cuff may overlap the tip 224 and the annealed section of the sheath shaft 232 to strengthen the butt joint. Coupling the tip 224 to the sheath shaft 232 may be accomplished, for example, via loading the components on a low surface friction mandrel (not necessarily the same low surface friction mandrel discussed above, though it may be). An FEP shell (not necessarily the same FEP shell discussed above) may encompass the tip 22 and the sheath shaft 232. Heat may cause the FEP shell to shrink and apply pressure while the tip 224 melts to the sheath shaft 232.

The sheath shaft 232 with an overmolded tip 224. The tip 224 may couple to the sheath shaft 232 at a distal end and may be coupled via an overmolding process. Similarly, the hub 210 may be overmolded at a proximal end of the sheath shaft 232. In some embodiments the tip 224 may be more radiopaque than other components to facilitate imaging.

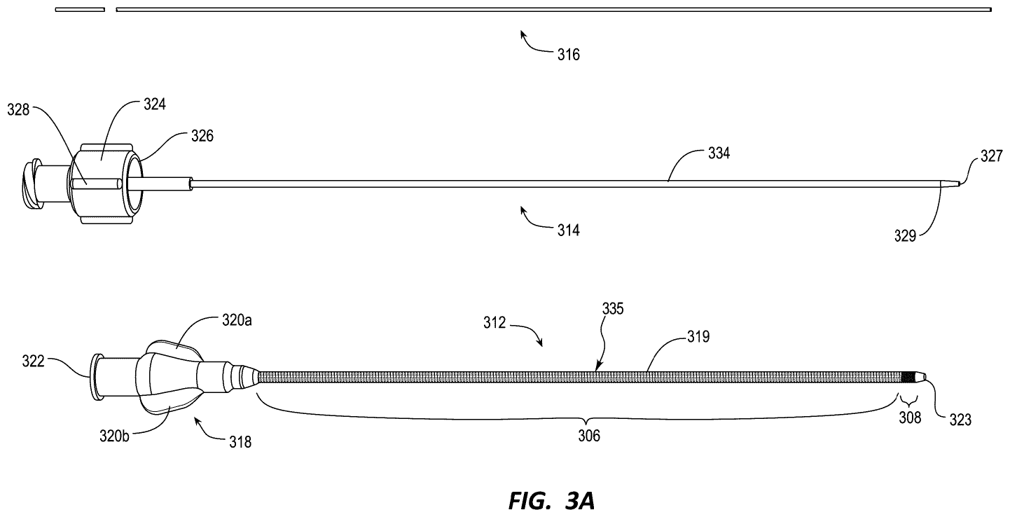

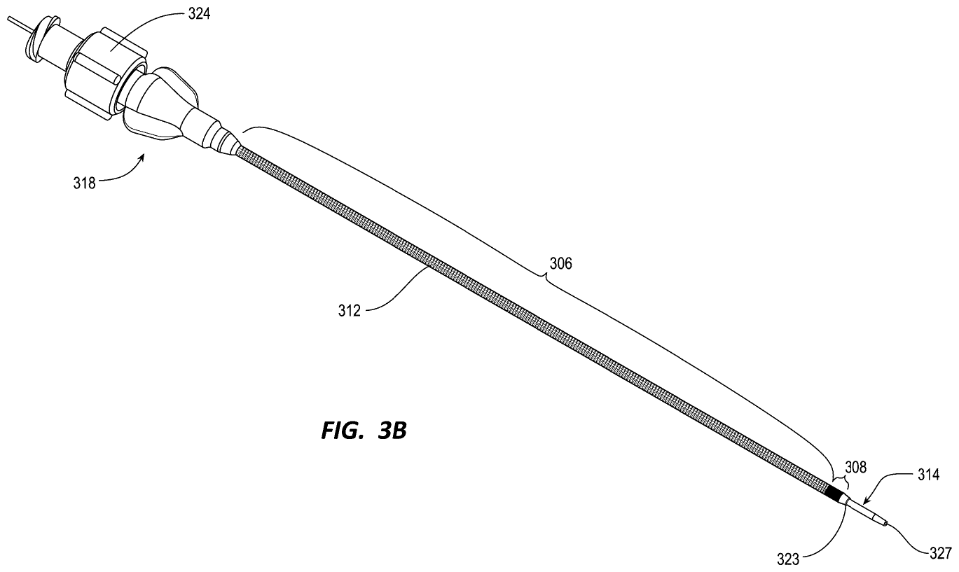

Other embodiments of introducer sheaths including a shaft having a coiled wire frame formed according embodiments of the catheter coil winding system 110 described above are also disclosed herein. FIG. 3A is a perspective view of an introducer sheath assembly 300 having a catheter sheath 312 (e.g. sheath shaft), a dilator 314, and a guidewire 316. The catheter sheath 312 includes a sheath tube 319 with the coiled wire frame 335 disposed therein. For example, the catheter sheath 312 may include the coiled wire frame 335 having an annealed segment and disposed between a liner and a jacket. Unless otherwise noted, the coiled wire frame 335 may include any aspect (e.g. materials or shape) of the coiled wire frame 134, 234. Furthermore, the liner and the jacket of the catheter sheath 312 may include any aspect (e.g., materials or shape) of the liner 102 and/or the jacket 106.

In some embodiments, the coiled wire frame 335 includes wire disposed more densely in the distal portion 308 than the proximal portion 306 of the wire frame 335. The wire frame 335, including the distal portion 308 and the proximal portion 306, may include any aspect of other wire frames described herein, such as the materials, dimensions, configurations, and positioning of: the coiled wire frame 134 including the proximal portion 136 and the distal portion 136 or the coiled wire frame 234 including the distal portion 236 and the proximal portion 238. Furthermore, the introducer sheath assembly 300.

The Introducer sheath assembly 300 is utilized to facilitate the introduction of a guidewire or catheter into the vasculature or other body cavity of a patient. Micropuncture catheters have been developed to provide a relatively smaller access puncture into the vasculature of the patient. Micropuncture introducer sheath assemblies include an introducer sheath which allows larger diameter guidewires to be introduced into the vasculature of the patient through the smaller access opening.

In the illustrated embodiment, introducer sheath assembly 300 comprises a catheter sheath 312, a dilator 314, and a guidewire 316. During a procedure in which introducer sheath assembly 300 is utilized, catheter sheath 312 and dilator 314 will typically be coupled together allowing the introducer sheath assembly 300 to be threaded into the patient. Dilator 314 is inserted along the length of catheter sheath 312 to provide rigidity and stiffness to facilitate the insertion of catheter sheath 312 into the patient. To insert catheter sheath 312 and dilator 314 into the patient, guidewire 316 is first inserted into the desired position within the patient through an access needle which has been inserted through the skin of the patient and into a vein, artery, or body cavity. Once guidewire 316 has been threaded through the access needle and into the patient, the needle can be withdrawn leaving guidewire 316 in place within the patient.

The relatively small diameter of the components of a micropuncture introducer sheath assembly 300 allow for a relatively small access puncture into the patient. For example, typically the access needle utilized with guidewire 316 can be a 21 gauge access needle or smaller. This allows a smaller guidewire 316 to be utilized. For example, guidewire 316 can be a 0.018″ guidewire or smaller. By utilizing such small access needles and guidewires with introducer sheath assembly 300, the original access puncture to the patient's skin, body cavity, or vasculature is quite small. The small access puncture facilitates a shorter recovery time, less trauma to the patient, and while abbreviating the length of bleeding from the access puncture and/or the vasculature of the patient.

Once guidewire 316 is positioned in the desired location within the patient, dilator 314 is threaded over guidewire 316. As previously discussed, dilator 314 is positioned within catheter sheath 312 such that when guidewire 316 is threaded along the length of dilator 314, guidewire 316 is also threaded along the length of catheter sheath 312. Dilator 314 and catheter sheath 312 are then advanced along the length of guidewire 316 through the access puncture in the patient's skin and into the desired position within the patient's body. The configuration of the tip of catheter sheath 312 and dilator 314 result in little tearing or trauma at the access puncture. Instead, catheter sheath 312 and dilator 314 results in stretching of the access puncture in a manner such that the size of the access puncture quickly returns to the original puncture size allowing for quicker healing at the access puncture site. Once dilator 314 and catheter sheath 312 have been inserted into the patient, guidewire 316 is withdrawn from the patient. Subsequently, dilator 314 will be withdrawn from the catheter sheath 312 allowing for the practitioner to access the body cavity or vasculature of the patient through catheter sheath 312.

In one embodiment, a larger diameter guidewire can be threaded through catheter sheath 312 and into the vasculature of the patient without requiring a larger access puncture typically required when a larger guidewire is inserted directly through an access needle. For example, such larger diameter guidewires can be a 0.038″ diameter guidewire which requires an approximately 0.040″ diameter puncture needle to be inserted into the patient. Utilizing a 0.040″ diameter puncture needle instead of the exemplary 21 gauge needle which is utilized in connection with catheter sheath 312 results in an access puncture that can be more than twice the size of the puncture required when utilizing introducer sheath assembly 300.

In the illustrated embodiment, catheter sheath 312 comprises a sheath hub 318 and a sheath tube 319. Sheath tube 319 is coupled to sheath hub 318 in a manner that allows for desired operation of sheath tube 319 relative to sheath hub 318. Additionally, sheath tube 319 is in fluid communication with sheath hub 318 allowing access to sheath tube 319 along the length of sheath hub 318.

In the illustrated embodiment, sheath hub 318 comprises wings 320 a, b and a lower coupler 322. Wings 320 a, b facilitate manipulation of catheter sheath 312 and introducer sheath assembly 300 by providing a gripping point for the practitioner during utilization of the introducer sheath assembly 300. Lower coupler 322 is positioned at the proximal end of catheter sheath 312. Lower coupler 322 allows for coupling of the dilator 314 to the catheter sheath 312 during operation of the introducer sheath assembly.

Sheath tube 319 includes a sheath tip 323 positioned at the distal end of the sheath tube 319. Sheath tip 323 is slightly tapered allowing for insertion of the catheter sheath into the access puncture. Sheath tube 319 typically has a resilient and somewhat flexible configuration allowing for introduction of larger diameter guidewires along the length of catheter sheath 312. The resilient nature of sheath tube 319 also permits expansion of the access puncture in the patient's skin while protecting the patient from damage as the guidewire is inserted.

Dilator 314 may comprise a dilator hub 324 and a stiffener tube. Stiffener tube provides additional rigidity and strength to dilator sleeve 334 as introducer sheath assembly 300 is inserted into the patient. Dilator hub 324 allows for manipulation of stiffener tube while also allowing for coupling of the dilator 314 to the catheter sheath 312 during utilization of the introducer sheath assembly 300. In the illustrated embodiment, dilator hub 324 comprises a lower coupler 326 and gripping members 328. Lower coupler 326 is positioned on the distal side of dilator hub 324 allowing for mating engagement of dilator hub 324 and sheath hub 318. Gripping members 328 are positioned on the outside diameter of dilator hub 324 allowing for gripping of the dilator hub 324 by the practitioner. By gripping the dilator hub 324, the practitioner can manipulate the dilator 314 to secure the dilator hub 324 to the sheath hub 318. Additionally, gripping members 328 of dilator hub 324 allow the practitioner to manipulate the introducer sheath assembly 300 during the course of the procedure.

Dilator sleeve 334 includes a dilator tip 327. Dilator tip 327 comprises a somewhat resilient member which is configured to prevent damage to the patient tissue as introducer sheath assembly 300 is threaded along the length of guidewire 316 and into the patient. Dilator tip 327 is somewhat more resilient and deformable than stiffener tube. This is due to the fact that the stiffener tube is positioned within dilator sleeve 334 but does not extend into dilator tip 327.

Transition 329 represents the point at which the stiffener tube terminates and dilator tip 327 begins. In the illustrated embodiment, transition 329 is positioned at a point that is more proximal than traditional transition points on stiffened micropuncture catheters. This allows for transition 329 to be positioned proximally to the sheath tip 323 of catheter sheath 312 when dilator 314 is threaded along the length of catheter sheath 312 and dilator hub 324 is coupled to sheath hub 318. Additionally, dilator tip 327 has a greater length from transition 329 to the distal tip of dilator tip 327. The greater length of dilator tip 327 allows a portion of dilator lip 327 to be positioned proximally to sheath tip 323 and within the catheter sheath 312. Additionally, a portion of dilator tip 327 can be positioned outside of catheter sheath 312 and distally to sheath lip 323.

The positioning of transition 329 within catheter sheath 312 allows catheter sheath 312 to provide strain relief to dilator tip 327 subsequent to lateral movement of the dilator tip 327. Positioning transition 329 within catheter sheath 312 relieves the strain that would normally be carried primarily at transition 329. Such strain is caused due to the stiffer configuration of dilator sleeve 334, which is co-extensive with the stiffener tube, and the more flexible nature of dilator tip 327. By providing strain relief subsequent to lateral movement of dilator tip 327, potentially damaging forces at transition 329 are dissipated. By dissipating such forces, kinking, buckling, or bending of catheter tip 327 at transition 329 is minimized in a manner that could result in the failure of dilator tip 27 during the procedure. In other words, minimizing the potential for damage at transition 329 provides for continued integrity of dilator 314 during the course of an insertion procedure.

As will be appreciated by those skilled in the art, a variety of types and configurations of introducer sheath assemblies can be utilized without departing from the scope or spirit of the present invention. For example, in one embodiment, the transition point is positioned at a traditional location along the length of the dilator. An elongate catheter sheath is provided such that the sheath tip is positioned distally to the transition. In another embodiment, a standard sized catheter sheath is utilized with a dilator having a shorter stiffener. An elongated dilator tip is provided such that the transition is moved proximally behind the tip of the catheter sheath. In yet another embodiment, a combination of an elongated catheter sheath, an elongated dilator tip, and a proximally positioned transition is utilized to provide a strengthened dilator tip. In yet another embodiment, the dilator hub is secured to the sheath hub utilizing other than a lower coupling. In yet another embodiment, a single hub is provided instead of two hubs.

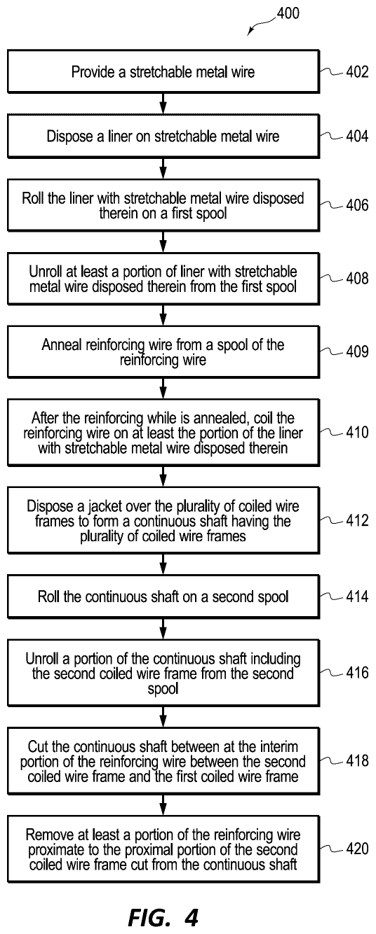

Turning ahead in the drawings, FIG. 4 is a flow diagram of an example of a method 400 of manufacturing a sheath, such as an introducer sheath, according to one embodiment. The steps shown in FIG. 4 may be optional and not necessarily including in each process. Further, various steps may be completed in different sequences from those shown in FIG. 4.

In some embodiments, the method 400 may include providing 402 a stretchable wire 104. The method 400 also may include providing a liner 104 having the metal wire 102 disposed therein or disposing 404 a liner 102 on the stretchable metal wire 104. For example, the method 400 may include disposing the liner 102 on the metal wire 104. In some embodiments, disposing 404 the liner 102 on the stretchable metal wire 104 includes sliding the liner 102 onto the stretchable metal wire 104. In some embodiments, the method 400 may include rolling 406 the liner 102 with stretchable metal wire 104 disposed therein (e.g., the elongate member 100) on a first spool 120. For example, at least 1000 feet of the liner 102 with the stretchable wire disposed therein may be rolled onto the first spool 120. The method 400 also may include unrolling 408 at least a portion of the liner 102 with stretchable metal wire 104 disposed therein from the first spool 120.

In some embodiments, the method 400 includes unrolling reinforcing wire 114 from a spool 112 prior to annealing a segment of the reinforcing wire 114. The method 400 also includes, after unrolling the reinforcing wire 114 from the spool 112, annealing 409 a segment of the reinforcing wire 114 such that the annealed segment of the reinforcing wire 114 is between two unannealed regions of the reinforcing wire 114. In some embodiments, annealing 409 the segment of the reinforcing wire 114 includes activating one or more electrodes positioned along the reinforcing wire 114 between the spool 112 and the liner 102 (e.g., the elongate member) to heat the segment of the reinforcing wire 114, and then deactivating the one or more electrodes such that the two unannealed regions of the reinforcing wire 114 bordering the segment of reinforcing wire 114 are not heated by the one or more electrodes. As described above, the one or more electrodes may be secured to one or more pulleys 116a-116d positioned along the reinforcing wire 114 between the spool 112 and the liner 102. Annealing 409 the segment of the reinforcing wire 114 may include annealing the segment of the reinforcing wire 144 between two pulleys (e.g., 116a, 116b) positioned along the reinforcing wire between the spool 112 and the liner 102, with the one or more electrodes being secured to at least one pulley (e.g., both) of the two pulleys. In some embodiments, annealing 409 the segment of the reinforcing wire 114 includes bringing the segment of the reinforcing wire 114 to at least about 75% of the melting temperature of the reinforcing wire 114, such as about 75% to about 85%, about 75% to about 80%, about 77.5% to about 82.5%, about 80% to about 85%, about 75%, about 77.5%, about 80%, about 82.5%, or about 85% of the melting temperature of the reinforcing wire 114.

In some embodiments, the method 400 includes, after annealing the segment of the reinforcing wire, coiling 410 the reinforcing wire 114 over the liner to form a wire frame 134 having a distal portion 136 and a proximal portion 138. The wire may be coiled 410 or disposed more densely in the distal portion 136 than in the proximal portion 138, and the segment of the reinforcing wire 114 that is annealed may be at least proximate to the distal portion 136 of the wire frame 134. In some embodiments, the annealed segment of the reinforcing wire 114 is coiled over the liner 102 within about 60 seconds of annealing the segment of the reinforcing wire 114.

In many embodiments, annealing 409 the segment of the reinforcing wire 114 reduces the at least one (e.g., both) of the hardness and/or spring constant in the coils of the wire frame 134 that include the segment of the reinforcing wire 114 that is annealed. For example, the hardness and/or spring constant in the coils of the wire frame 134 that include the segment of the reinforcing wire 114 that is annealed may be reduced about 10% to about 20%, about 10% to about 15%, about 12.5% to about 17.5%, about 15% to about 20%, at least about 10%, at least about 12.5%, at least about 15%, at least about 17.5%, about 10%, about 12.5%, about 15%, about 17.5%, or about 20% relative to portions of the wire frame 134 that are not annealed.

In some embodiments, coiling 410 the reinforcing wire 114 over the liner 102 includes coiling the reinforcing wire 114 on at least the portion of the liner 102 unrolled from the first spool 120 to form (1) a plurality of coiled wire frames 134 each having the proximal portion 138 and the distal portion 136 having the reinforcing wire 114 coiled more densely than the proximal portion 138, and also (2) an interim portion 132 of the reinforcing wire 114 coiled between the distal portion 136 of a first wire frame 134′ of the plurality of wire frames 134 and a proximal portion 136 of a second wire frame 134″ of the plurality of wire frames 134 adjacent to the first wire frame 134′. The distal portion 136 of the first wire frame 134′ is spaced from the proximal portion 138 of the second wire frame 134″ by the interim portion 132. In many embodiment, coiling 410 the reinforcing wire 114 over the liner 102 of the elongate member 100 includes rotating at least one (e.g., all) of the first spool 112 and/or the one or more pulleys 116a-d around the liner 102 of the elongate member 100 to coil the reinforcing wire 114 over the liner 102.

In some embodiments, the method 400 includes pulling the elongate member 100 from the first spool 120 as the reinforcing wire 114 is being coiled around a portion of the elongate member 100 (e.g., as at least one of the first spool 112 and/or the one or more pulleys 116a-d are rotated around the elongate member 100). For example, the method 400 may include pulling the elongate member 100 from the first spool 120 effective to form the proximal portion 138 and the distal portion 136 of the wire frame 134 on the elongate member 100. More specifically, in the method 400, the elongate member 100 may be pulled from the first spool 120 at a first rate when the proximal portion 138 is being formed and a second rate slower than the first rate when the distal portion 136 is being formed. Pulling the elongate member 100 from the first spool 120 at the first rate and the second rate is effective for form the distal portion 136 having the reinforcing wire 114 coiled more densely thereon than the proximal portion 138 of the wire frame 134.

In some embodiments, the method 400 includes pulling the elongate member 100 from the first spool 120 at a third rate to form an interim portion 132 of reinforcing wire 114 on the elongate member 100. In the method 400, the third rate may be faster than the first rate and the second rate such that the reinforcing wire 114 is coiled less densely (e.g., wider pitch) than both the proximal portion 138 and the distal portion of the coiled wire frame 134.

In some embodiments, the method 400 may include rotating the second spool 130 around the axis of the second spool 130 at different rates effective pull the elongate member 100 from the first spool 120 at the first rate, the second rate, and/or the third rate to form the proximal portion 138, the distal portion 136, and the interim portion 132 on the elongate member 100. The method 400 also may include heating the liner 102 having the coiled wire frame 134 disposed thereon prior to or as the liner 102 and the coiled wire frame 134 are rolled onto the second spool 130. This heating may help tack the coiled wire frame 134 to the surface of the liner 102.

In some embodiments, the method 400 may include rolling 411 the plurality of coiled wire frames 134 on the liner 102 on a second spool 130. The method also may include unrolling 412 at least some of the plurality of coiled wire frames 134 on the liner 102 from the second spool 120 and securing or disposing 413 a jacket 106 over the unrolled at least some of plurality wire frames 134 (e.g. at least the first coiled wire frame 134′ and the second coiled wire frame 134″) and the liner 102 to form a continuous shaft. Securing or disposing 413 the jacket 406 over the unrolled wire frames 134 and the liner 102 may include sliding the jacket 106 over the unrolled wire frames 134 and the liner 102, and then heat shrinking the jacket 106 over the unrolled wire frames 134 and the liner 102. In some embodiments, securing the jacket 106 over the unrolled wire frames 134 and the liner 102 includes securing a jacket 106 over the unrolled wire frames 134 and the interim portion 132 of the reinforcing wire 114 coiled on the portion of the liner 102 to form a continuous shaft 140 having the at least some of the plurality of wire frames 134. In some embodiments, the liner 102 may be heated before the jacket 106 is disposed over the multiple wire frames and the liner 102.

The method 400 may optionally include, before disposing 413 the jacket over the wire frame 134 and the liner 102, sliding a heat shrink material over at least some of the reinforcing wire coiled at a proximal region of the liner 102, and also heat shrinking the heat shrink material over the at least some of the reinforcing wire coiled at the proximal region of the liner 102. In some embodiments, the method 400 includes reflowing the liner 102 and the jacket 106 after securing the jacket 106 over the multiple wire frames 134 and the liner 102.

The method 400 also may include rolling 414 the continuous shaft 140 onto a third spool. The method 400 also may include unrolling 415 a portion of the continuous shaft 140 comprising multiple wire frames 134 including at least the first coiled wire frame 134′ and the second coiled wire frame 134″ from the third spool. The method 400 also may include cutting 416 the unrolled portion of the continuous shaft 140 comprising the multiple wire frames 134 (e.g. at least the first coiled wire frame 134′ and the second coiled wire frame 134″) from the third spool. For example, the portion cut from the third spool may be approximately three feet of continuous shaft 140 including multiple wire frames 134 between the liner 102 and the jacket 106.

The method 400 also may include removing 417 the metal wire 104 from the liner 102 after cutting the portion of the continuous shaft 140 from the third spool. For example, the method 400 may include stretching the metal wire 104 in the cut portion of continuous shaft 140 while the metal wire 104 is in the liner 102 to narrow the metal wire 104, and then pulling the metal wire 104 from the liner 102. In some embodiments, the method 400 includes removing 420 at least a portion of the reinforcing wire 114 from the first sheath proximate to the distal portion 136 of the second coiled wire frame 134″ cut from the continuous shaft. Removing 420 at least a portion of the reinforcing wire 114 from the first sheath proximate to the distal portion 136 of the second coiled wire frame 134″ may include includes laser cutting or laser ablating the wire frame 134″ about 0.5 mm to about 2 mm from a terminating end of the first sheath.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the term “near.” For each such reference, it is to be understood that, in some embodiments, the value, feature, or characteristic may be specified without approximation. For example, where qualifiers such as “near” and “approximately” are used, these terms include within their scope the qualified words in the absence of their qualifiers. For example, where the term “approximately aligned” is recited with respect to a feature, it is understood that in further embodiments, the feature can have a precisely aligned configuration.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A method of manufacturing a sheath, the method comprising:
providing a liner;
annealing a segment of a reinforcing wire such that the segment is between two unannealed regions of the reinforcing wire;
after annealing the segment of the reinforcing wire, coiling the reinforcing wire over the liner to form a wire frame having a distal portion and a proximal portion, the wire being disposed more densely in the distal portion than in the proximal portion and the segment of the reinforcing wire that is annealed is at least proximate to the distal portion of the wire frame; and
securing a jacket over the wire frame and the liner.

2. The method of claim 1, further comprising unrolling the reinforcing wire from a spool prior to annealing the segment of the reinforcing wire.

3. The method of claim 2, wherein annealing the segment of the reinforcing wire includes:
activating one or more electrodes positioned along the reinforcing wire between the spool and the liner to heat the segment of the reinforcing wire; and
deactivating the one or more electrodes such that the two unannealed regions of the reinforcing wire bordering the segment are not heated by the one or more electrodes.

4. The method of claim 3, wherein the one or more electrodes are secured to one or more pulleys positioned along the reinforcing wire between the spool and the liner.

5. The method of claim 4, wherein annealing the segment of the reinforcing wire includes annealing the segment of the reinforcing wire between two pulleys positioned along the reinforcing wire between the spool and the liner, the one or more electrodes being secured to at least pulley of the two pulleys.

6. The method claim 1, wherein annealing the segment of reinforcing wire reduces the hardness and/or spring constant by at least about 10% in coils of the wire frame that include the segment of reinforcing wire that is annealed relative to the hardness and/or spring constant of coils of the wire frame that include the two unannealed regions.

7. The method of claim 1, further comprising, before securing the jacket over the coiled wire frame and the liner:
sliding a heat shrink material over at least some of the reinforcing wire coiled at a proximal region of the liner; and
heat shrinking the heat shrink material over the at least some of the reinforcing wire coiled at the proximal region of the liner.

8. The method of claim 1, wherein securing the jacket over the coiled wire frame and the liner includes sliding the jacket over the wire frame and heat shrinking the jacket over the wire frame.

9. The method of claim 1, further comprising reflowing the liner and the jacket after securing the jacket over the coiled wire frame and the liner.

10. The method of claim 1, further comprising:
wherein providing the liner includes providing of a first spool having the liner rolled thereon, the liner having a metal wire disposed therein;
unrolling at least a portion of the liner from the first spool;
wherein coiling the reinforcing wire over the liner includes coiling the reinforcing wire on at least the portion of the liner unrolled from the first spool to form (1) a plurality of coiled wire frames each having the proximal portion and the distal portion having the reinforcing wire coiled more densely than the proximal portion, and (2) an interim portion of the reinforcing wire coiled between the distal portion of a first wire frame of the plurality of wire frames and a proximal portion of a second wire frame of the plurality of wire frames adjacent to the first wire frame such that the distal portion of the first wire frame is spaced from the proximal portion of the second wire frame; and
wherein securing the jacket over the wire frame and the liner includes securing a jacket over the plurality of coiled wire frames and the interim portion of the reinforcing wire coiled on the portion of the liner to form a continuous shaft having the plurality of coiled wire frames.

11. The method of claim 10, further comprising:
before securing the jacket over the plurality of coiled wire frames and the liner, rolling the plurality of coiled wire frames and the liner onto a second spool;
before securing the jacket over the plurality of coiled wire frames and the liner, unrolling the plurality of coiled wire frames and the liner from the second spool; and
after securing the jacket over the plurality of coiled wire frames and the liner, rolling the continuous shaft on a third spool.

12. The method of claim 11, further comprising:
unrolling a portion of the continuous shaft including the second coiled wire frame from the third spool; cutting the continuous shaft at the interim portion of the reinforcing wire between the second coiled wire frame and the first coiled wire frame to form a first sheath.

13. The method of claim 12, further comprising removing at least a portion of the reinforcing wire from the first sheath proximate to the distal portion of the second coiled wire frame cut from the continuous shaft.

14. The method of claim 13, wherein removing at least a portion of the reinforcing wire from the first sheath proximate to the distal portion of the second coiled wire frame includes laser cutting the wire frame about 0.5 mm to about 2 mm from a terminating end of the first sheath.

15. The method of claim 10, wherein providing of a first spool having a liner rolled thereon includes:
providing a silver plated copper wire;
disposing the liner on the silver plated copper wire such that the liner has the silver plated copper wire disposed therein; and
rolling the liner having the silver plated copper wire disposed therein around the first spool.

16. The method of claim 10, further comprising removing the metal wire from the liner after securing the jacket over the plurality of coiled wire frames and the interim portion of the reinforcing wire coiled on the portion of the liner.

17. A system for annealing segments of a reinforcing wire, the system comprising:
a spool holder configured to hold a spool of reinforcing wire;
a assembly configured to hold and/or rotate an elongate member, the winding assembly being positioned to have the reinforcing wire from the spool of reinforcing wire coiled around at least a portion of the elongate member;
an annealing assembly configured selectively anneal segments of the reinforcing wire between the spool holder and the elongate member held by the assembly.

18. The system of claim 17, wherein the annealing assembly includes:
one or more pulleys positioned between the spool holder and the elongate member held by the assembly and configured to have the reinforcing wire wind around a portion of each of the one or more pulleys before being coiled around the elongate member;
an electrode secured to at least one pulley of the one or more pulleys; and
an actuator configured to activate and deactivate the electrode effective to anneal a segment of the reinforcing wire before the segment of the reinforcing wire is coiled around at least the portion of the elongate member.

19. The system of claim 18, wherein the one or more pulleys include two pulleys, the electrode is secured to at least one pulley of the two pulleys, and the actuator is configured to activate and deactivate the electrode effective to anneal the segment of the reinforcing wire between the two pulleys before the segment of the reinforcing wire is coiled around at least the portion of the elongate member.

20. The system of claim 17, further comprising the spool of reinforcing wire and the elongate member.
